(19) United States
(12) Patent Application Publication
(10) Pub. No.: US 2015/0093403 A1
(43) Pub. Date: Apr. 2, 2015

(54) CANINE CIRCOVIRUS SEQUENCES AND USES THEREOF

(71) Applicant: THE TRUSTEES OF COLUMBIA UNIVERSITY IN THE CITY OF NEW YORK, New York, NY (US)

(72) Inventors: W. Ian Lipkin, New York, NY (US); Amit Kapoor, New York, NY (US); Edward J. Dubovi, Ithaca, NY (US)

(21) Appl. No.: 14/390,668

(22) PCT Filed: Apr. 3, 2013
(86) PCT No.: PCT/US13/35120

§ 371 Date: Oct. 3, 2014

Related U.S. Application Data
(60) Provisional application No. 61/619,769, filed on Apr. 3, 2012.

(51) Int. Cl. C12N 7/00; C12Q 1/70; C07K 14/005; C07K 16/08

(52) U.S. Cl. CPC C12N 7/00; C07K 16/081; C12Q 1/701; C07K 14/005; C12N 2750/00021; C12N 2750/00033; C12N 2750/00071
USPC 424/186.1; 536/23.72; 435/194; 536/23.2; 530/350; 530/387.9; 435/5; 536/24.5; 514/44 A; 435/235.1

(57) ABSTRACT

The invention is directed to a isolated a canine circoviruses associated with canine respiratory and gastrointestinal disease, and isolated nucleic acids sequences and polypeptides thereof. The invention also relates to antibodies against antigens from canine circoviruses. The invention also relates to iRNAs which target nucleic acid sequences of the canine circovirus. The invention is related to methods for detecting the presence or absence of canine circoviruses in an animal. The invention is also related to immunogenic compositions for inducing an immune response against canine circoviruses in an animal.

This application claims the benefit of and priority to U.S. provisional patent application Ser. No. 61/619,769 filed Apr. 3, 2012, the disclosure of which is hereby incorporated by reference in its entirety for all purposes

This invention was made with government support under AI090196, AI081132, AI079231, AI57158, AI070411 and EY017404 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### BACKGROUND

The family Circoviridae contains three genera, Circovirus, Cyclovirus and Gyrovirus. There is a need for a diagnostic test, an immunogenic composition and a method of treating animals (e.g. dogs) having circoviral infections. This invention addresses these needs.

### SUMMARY OF THE INVENTION

The invention is related to a novel canine circovirus associated with canine respiratory and gastrointestinal disease, and isolated nucleic acids sequences and peptides thereof. The invention is also related to antibodies against antigens derived from the canine circovirus. The invention is also related to iRNAs which target nucleic acid sequences of the canine circovirus. The invention is related to methods for detecting the presence or absence of canine circovirus an animal. The invention is also related to immunogenic compositions for inducing an immune response against canine circovirus an animal.

In certain aspects, the invention relates to an isolated nucleic acid having the sequence of SEQ ID NO: 1.

In certain aspects, the invention relates to an isolated nucleic acid having at least about 60% sequence identity to SEQ ID NO: 1.

In certain aspects, the invention relates to an isolated nucleic acid comprising at least 10 consecutive nucleotides from SEQ ID NO: 1.

In certain aspects, the invention relates to an isolated nucleic acid which comprises at least 10 consecutive nucleotides of a sequence having at least about 60% identity to SEQ ID NO: 1.

In certain aspects, the invention relates to an isolated nucleic acid which comprises consecutive nucleotides having a sequence complementary to an isolated nucleic acid which comprises at least 10 consecutive nucleotides of SEQ ID NO: 1 or an isolated nucleic acid which comprises at least 10 consecutive nucleotides of a sequence having at least about 60% identity to SEQ ID NO: 1.

In certain aspects, the invention relates to an isolated polypeptide having the sequence of any of SEQ ID NOs: 2-4.

In certain aspects, the invention relates to an isolated polypeptide having at least about 80% sequence identity to any of SEQ ID NO: 2-4.

In certain aspects, the invention relates to an isolated polypeptide comprising at least 8 consecutive amino acids of any of SEQ ID NOs 2-4.

In certain aspects, the invention relates to an isolated polypeptide comprising at least 8 amino acids having at least about 80% identity to the sequence of any of SEQ ID NOs 2-4.

In certain aspects, the invention relates to an isolated nucleic acid encoding a polypeptide having the sequence of any of SEQ ID NOs: 2-4, a polypeptide having at least about 80% sequence identity to any of SEQ ID NO: 2-4, a polypeptide comprising at least 8 consecutive amino acids of any of SEQ ID NOs 2-4, or a polypeptide comprising at least 8 amino acids having at least about 80% identity to the sequence of any of SEQ ID NOs 2-4

of any of claims 6-9.

In certain aspects, the invention relates to an isolated antibody that specifically binds to a polypeptide having the sequence of any of SEQ ID NOs: 2-4, a polypeptide having at least about 80% sequence identity to any of SEQ ID NO: 2-4, a polypeptide comprising at least 8 consecutive amino acids of any of SEQ ID NOs 2-4, or a polypeptide comprising at least 8 amino acids having at least about 80% identity to the sequence of any of SEQ ID NOs 2-4.

In certain aspects, the invention relates to an immunogenic composition comprising at least about 24 consecutive nucleotides from a nucleic acid having the sequence of SEQ ID NO: 1 or from a nucleic acid having at least about 60% sequence identity to SEQ ID NO: 1.

In certain aspects, the invention relates to an immunogenic composition comprising at least about 8 consecutive amino acids from a polypeptide having the sequence of any of SEQ ID NOs: 2-4, or from a polypeptide having at least about 80% sequence identity to any of SEQ ID NO: 2-4.

In certain aspects, the invention relates to a method of inducing an immune response in an animal, the method comprising administering an immunogenic composition comprising at least about 24 consecutive nucleotides from a nucleic acid having the sequence of SEQ ID NO: 1 or from a nucleic acid having at least about 60% sequence identity to SEQ ID NO: 1.

In certain aspects, the invention relates to a method of inducing an immune response in an animal, the method comprising administering an immunogenic composition comprising at least about 8 consecutive amino acids from a polypeptide having the sequence of any of SEQ ID NOs: 2-4, or from a polypeptide having at least about 80% sequence identity to any of SEQ ID NO: 2-4.

In certain aspects, the invention relates to a synthetic nucleic acid comprising at least about 10 nucleotides of a nucleic acid having the sequence of SEQ ID NO: 1 or from a nucleic acid having at least about 60% sequence identity to SEQ ID NO: 1.

In certain aspects, the invention relates to a synthetic nucleic acid comprising at least about 10 nucleotides complementary to a nucleic acid having the sequence of SEQ ID NO: 1 or from a nucleic acid having at least about 60% sequence identity to SEQ ID NO: 1.

In certain aspects, the invention relates to a method for determining the presence or absence of canine circovirus a biological sample, the method comprising: a) contacting nucleic acid from a biological sample with at least one primer which is a synthetic nucleic acid comprising at least about 10 nucleotides of a nucleic acid having the sequence of SEQ ID NO: 1 or from a nucleic acid having at least about 60% sequence identity to SEQ ID NO: 1, or a synthetic nucleic acid comprising at least about 10 nucleotides complementary to a nucleic acid having the sequence of SEQ ID NO: 1 or from a nucleic acid having at least about 60% sequence identity to SEQ ID NO: 1, b) subjecting the nucleic acid and the primer to amplification conditions, and c) determining the presence or absence of amplification product, wherein the presence of amplification product indicates the presence of RNA associated with of canine circovirus the sample.

In certain aspects, the invention relates to a primer set for determining the presence or absence of canine circovirus a biological sample, wherein the primer set comprises at least one synthetic nucleic acid sequence selected from the group consisting of: a) a synthetic nucleic acid comprising at least about 10 nucleotides of a nucleic acid having the sequence of SEQ ID NO: 1 or from a nucleic acid having at least about 60% sequence identity to SEQ ID NO: 1, and b) a synthetic nucleic acid comprising at least about 10 nucleotides complementary to a nucleic acid having the sequence of SEQ ID NO: 1 or from a nucleic acid having at least about 60% sequence identity to SEQ ID NO: 1.

In certain aspects, the invention relates to a method for determining whether or not a sample contains of canine circovirus, the method comprising: a) contacting a biological sample with an antibody that specifically binds to a polypeptide having the sequence of any of SEQ ID NOs: 2-4, a polypeptide having at least about 80% sequence identity to any of SEQ ID NO: 2-4, a polypeptide comprising at least 8 consecutive amino acids of any of SEQ ID NOs 2-4, or a polypeptide comprising at least 8 amino acids having at least about 80% identity to the sequence of any of SEQ ID NOs 2-4, and b) determining whether or not the antibody binds to an antigen in the biological sample, wherein binding indicates that the biological sample contains canine circovirus. In certain embodiments, the determining comprises use of a lateral flow assay or ELISA.

In certain aspects, the invention relates to a method for determining whether or not a biological sample has been infected by canine circovirus, the method comprising: a) determining whether or not a biological sample contains antibodies that specifically bind to a polypeptide having the sequence of any of SEQ ID NOs: 2-4 or polypeptide having at least about 80% sequence identity to any of SEQ ID NO: 2-4. In certain embodiments, the determining comprises determining whether the antibodies are IgY antibodies, wherein detection of IgY antibodies is indicative of a infection of the sample by a canine circovirus.

In certain aspects, the invention relates to an interfering RNA (iRNA) comprising at least 15 contiguous nucleotides complementary to a nucleic acid having the sequence of SEQ ID NO: 1 or a nucleic acid having at least about 60% sequence identity to SEQ ID NO: 1

In certain aspects, the invention relates to a method for reducing the levels of a canine circovirus protein in an animal, viral mRNA in an animal or viral titer in a cell of an animal, the method comprising administering to the animal an interfering RNA (iRNA) comprising at least 15 contiguous nucleotides complementary to a nucleic acid having the sequence of SEQ ID NO: 1 or a nucleic acid having at least about 60% sequence identity to SEQ ID NO: 1

In certain aspects, the invention relates to an isolated virus comprising at least 24 consecutive nucleotides from a nucleic acid having the sequence of SEQ ID NO: 1 or a nucleic acid having at least about 60% sequence identity to SEQ ID NO: 1

In certain aspects, the invention relates to an isolated virus comprising at least 8 consecutive amino acids from a polypeptide having the sequence of any of SEQ ID NOs: 2-4 or a polypeptide having at least about 80% sequence identity to any of SEQ ID NO: 2-4.

### BRIEF DESCRIPTION OF THE FIGURES

FIG. 1 shows the nucleic acid sequence of CaCV-CG214 (SEQ ID: NO 1) which is derived from a canine circovirus.

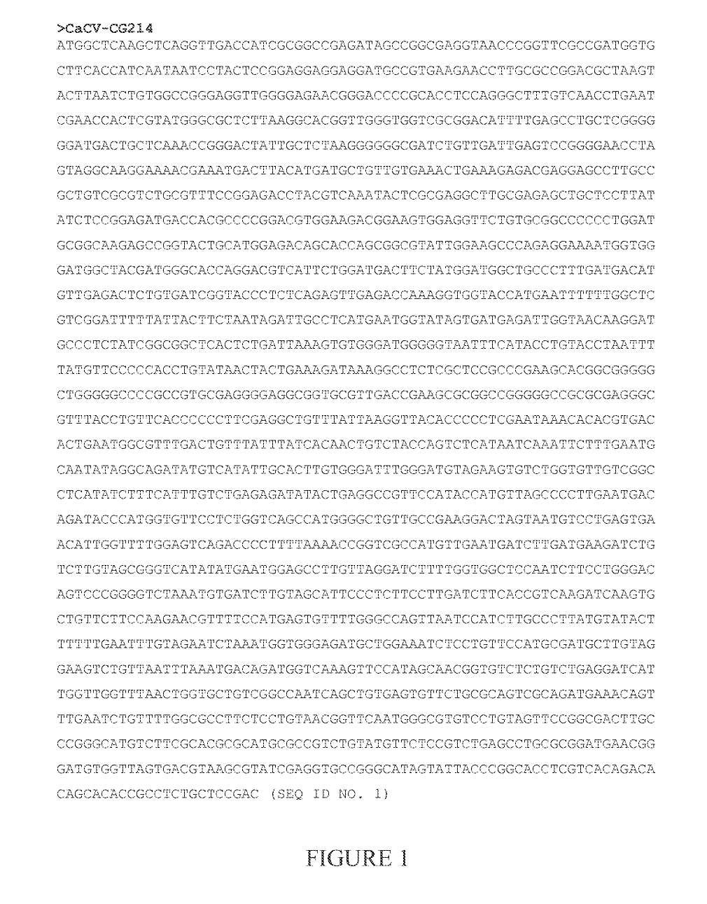

FIG. 2 shows the amino sequence of CaCV-Replicase-214 (SEQ ID NO: 2), CaCV-Capsid-214 (SEQ ID NO: 3). CaCV-ORF3-214 (SEQ ID NO: 4) which are derived from a canine circovirus.

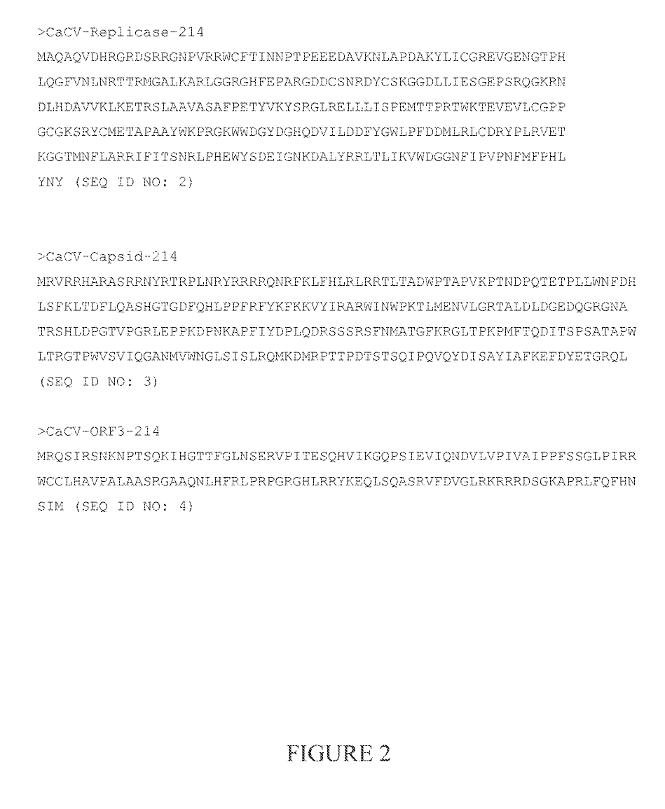

FIG. 3 shows a denogram showing the relationship between the new canine circovirus and other viruses. CaCV is distantly related to Procine circoviruses, known to cause diseases in pigs. CaCV replicase protein showed <55% protein identity with replicase protein of any known animal circovirus reported till date. CaCV capsid protein showed <25% protein identity with capsid protein of any known animal circovirus reported till date.

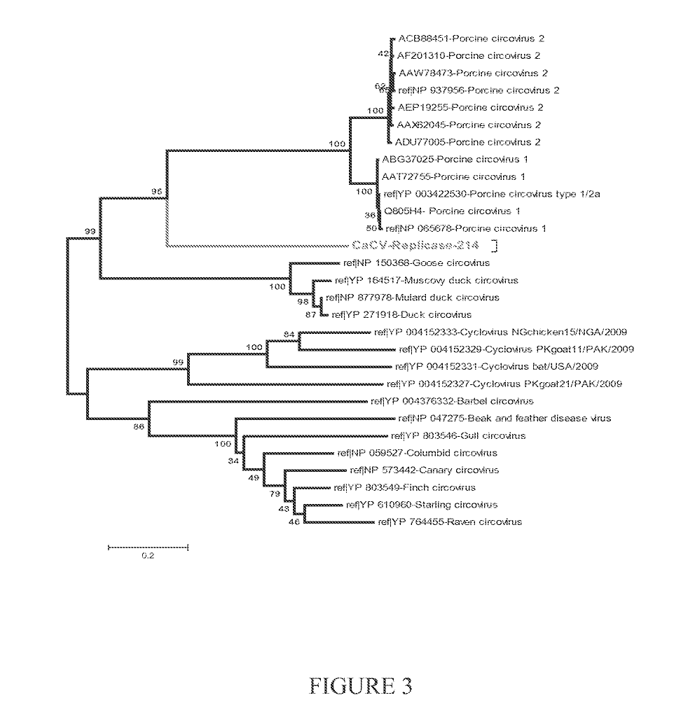

FIG. 4 the genome of CaCV-1 (strain—NY214) comprises 2063 nt as covalently closed circular DNA with a GC content of 51.7%.

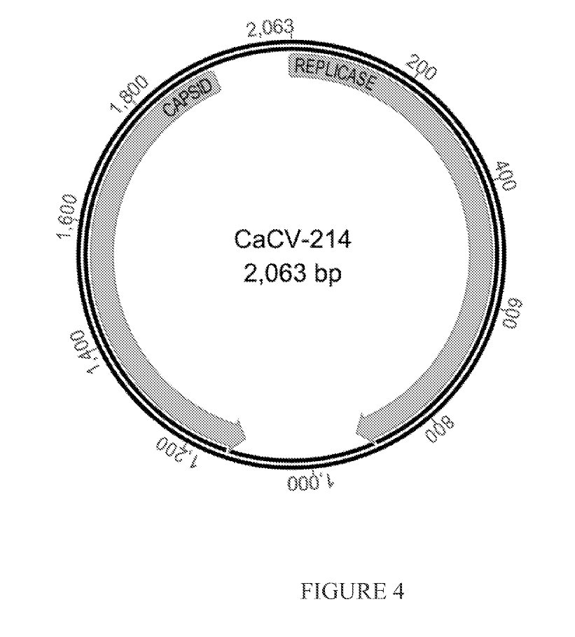

### DETAILED DESCRIPTION

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, “canine circovirus” refers to isolates of the canine circoviruses described herein.

As used herein, the term “animal” refers to a vertebrate, including, but not limited to canines (e.g. dogs). In one embodiment, an animal is a canine. In another embodiment, an animal is a feline. In certain embodiments, an animal can be a equine, sheep, cattle, poultry or humans,

As used herein, the term immunogenic composition refers to a composition capable of inducing an immunogenic response in an animal or a cell. As used herein, reference to an immunogenic composition can include a vaccine.

The present invention provides canine circovirus nucleic acid sequences. These nucleic acid sequences may be useful for, inter alia, expression of canine circovirus-encoded proteins or fragments, variants, or derivatives thereof, generation of antibodies against canine circovirus proteins, generation of primers and probes for detecting canine circovirus and/or for diagnosing canine circovirus infection, generating immunogenic compositions against canine circoviruses, and screening for drugs effective against canine circoviruses as described herein.

In certain aspects, the invention relates to variants of CaCV nucleic acid sequence having greater that 60% similarity to the sequence of SEQ ID NO: 1. In certain aspects, the invention relates to variants of CaCV amino acid sequence having greater that 80% similarity to the sequences of SEQ ID NO: 2-4.

In certain aspects, the invention is directed to isolated amino acid sequence variants of any one of SEQ ID NO: 2-4. Variants of SEQ ID NO: 2-4 include, but are not limited to, amino acid sequences having at least from about 50% to about 55% identity to that of SEQ ID NO: 2-4. Variants of SEQ ID NO: 2-4 include, but are not limited to, amino acid sequences having at least from about 55.1% to about 60% identity to that of SEQ ID NO: 2-4. Variants of SEQ ID NO: 2-4 include, but are not limited to, amino acid sequences having at least from about 60.1% to about 65% identity to that of SEQ ID NO: 2-4. Variants of SEQ ID NO: 2-4 include, but are not limited to, amino acid sequences having at least from about 65.1% to about 70% identity to that of SEQ ID NO: 2-4. Variants of SEQ ID NO: 2-4 include, but are not limited to, amino acid sequences having at least from about 70.1% to about 75% identity to that of SEQ ID NO: 2-4. Variants of SEQ ID NO: 2-4 include, but are not limited to, amino acid sequences having at least from about 75.1% to about 80% identity to that of SEQ ID NO: 2-4. Variants of SEQ ID NO: 2-4 include, but are not limited to, amino acid sequences having at least from about 80.1% to about 85% identity to that of SEQ ID NO: 2-4. Variants of SEQ ID NO: 2-4 include, but are not limited to, amino acid sequences having at least from about 85.1% to about 90% identity to that of SEQ ID NO: 2-4. Variants of SEQ ID NO: 2-4 include, but are not limited to, amino acid sequences having at least from about 90.1% to about 95% identity to that of SEQ ID NO: 2-4. Variants of SEQ ID NO: 2-4 include, but are not limited to, amino acid sequences having at least from about 95.1% to about 97% identity to that of SEQ ID NO: 2-4. Variants of SEQ ID NO: 2-4 include, but are not limited to, amino acid sequences having at least from about 97.1% to about 99% identity to that of SEQ ID NO: 2-4.

In certain aspects, the invention is directed to a canine circovirus isolated nucleic acid sequence as provided in SEQ ID NO: 1.

In certain aspects, the invention is directed to an isolated nucleic acid of SEQ ID NO: 1. In certain aspects, the invention is directed to an isolated nucleic acid complementary SEQ ID NO: 1.

In certain aspects, the invention is directed to isolated nucleic acid sequence variants of SEQ ID NO: 1. Variants of SEQ ID NO: 1 include, but are not limited to, nucleic acid sequences having at least from about 50% to about 55% identity to that of SEQ ID NO: 1. Variants of SEQ ID NO: 1 include, but are not limited to, nucleic acid sequences having at least from about 55.1% to about 60% identity to that of SEQ ID NO: 1. Variants of SEQ ID NO: 1 include, but are not limited to, nucleic acid sequences having at least from about 60.1% to about 65% identity to that of SEQ ID NO: 1. Variants of SEQ ID NO: 1 include, but are not limited to, nucleic acid sequences having at least from about 65.1% to about 70% identity to that of SEQ ID NO: 1. Variants of SEQ ID NO: 1 include, but are not limited to, nucleic acid sequences having at least from about 70.1% to about 75% identity to that of SEQ ID NO: 1. Variants of SEQ ID NO: 1 include, but are not limited to, nucleic acid sequences having at least from about 75.1% to about 80% identity to that of SEQ ID NO: 1. Variants of SEQ ID NO: 1 include, but are not limited to, nucleic acid sequences having at least from about 80.1% to about 85% identity to that of SEQ ID NO: 1. Variants of SEQ ID NO: 1 include, but are not limited to, nucleic acid sequences having at least from about 85.1% to about 90% identity to that of SEQ ID NO: 1. Variants of SEQ ID NO: 1 include, but are not limited to, nucleic acid sequences having at least from about 90.1% to about 95% identity to that of SEQ ID NO: 1. Variants of SEQ ID NO: 1 include, but are not limited to, nucleic acid sequences having at least from about 95.1% to about 97% identity to that of SEQ ID NO: 1. Variants of SEQ ID NO: 1 include, but are not limited to, nucleic acid sequences having at least from about 97.1% to about 99% identity to that of SEQ ID NO: 1. Programs and algorithms for sequence alignment and comparison of % identity and/or homology between nucleic acid sequences, or polypeptides, are well known in the art, and include BLAST, SIM alignment tool, and so forth.

In one embodiment, the invention is directed to an isolated nucleic acid sequence comprising from about 10 to about 50 consecutive nucleotides from any one of SEQ ID NO: 1 or a sequence complementary SEQ ID NO: 1. In one embodiment, the invention is directed to an isolated nucleic acid sequence comprising from about 10 to about 100 consecutive nucleotides from any one of SEQ ID NO: 1 or a sequence complementary SEQ ID NO: 1. In one embodiment, the invention is directed to an isolated nucleic acid sequence comprising from about 10 to about 200 consecutive nucleotides from any one of SEQ ID NO: 1 or a sequence complementary SEQ ID NO: 1. In one embodiment, the invention is directed to an isolated nucleic acid sequence comprising from about 10 to about 300 consecutive nucleotides from any one of SEQ ID NO: 1 or a sequence complementary SEQ ID NO: 1. In one embodiment, the invention is directed to an isolated nucleic acid sequence comprising from about 10 to about 400 consecutive nucleotides from SEQ ID NO: 1 or a sequence complementary SEQ ID NO: 1. In one embodiment, the invention is directed to an isolated nucleic acid sequence comprising from about 10 to about 500 consecutive nucleotides from any one of SEQ ID NO: 1 or a sequence complementary SEQ ID NO: 1. In one embodiment, the invention is directed to an isolated nucleic acid sequence comprising from about 10 to about 600 consecutive nucleotides from any one of SEQ ID NO: 1 or a sequence complementary SEQ ID NO: 1. In one embodiment, the invention is directed to an isolated nucleic acid sequence comprising from about 10 to about 700 consecutive nucleotides from any one of SEQ ID NO: 1 or a sequence complementary SEQ ID NO: 1. In one embodiment, the invention is directed to an isolated nucleic acid sequence comprising from about 10 to about 800 consecutive nucleotides from any one of SEQ ID NO: 1 or a sequence complementary SEQ ID NO: 1. In one embodiment, the invention is directed to an isolated nucleic acid sequence comprising from about 10 to about 800 or more consecutive nucleotides from any one of SEQ ID NO: 1 or a sequence complementary SEQ ID NO: 1.

In other aspects the invention is directed to isolated nucleic acid sequences such as primers and probes, comprising nucleic acid sequences of SEQ ID NO: 1. Such primers and/or probes may be useful for detecting the presence of the canine circovirus of the invention, for example in samples of bodily fluids such as blood, saliva, or urine from an animal, and thus may be useful in the diagnosis of canine circovirus infection. Such probes can detect polynucleotides of SEQ ID NO: 1 in samples which comprise canine circovirus represented by SEQ ID NO: 1. The isolated nucleic acids which can be used as primer and/probes are of sufficient length to allow hybridization with, i.e. formation of duplex with a corresponding target nucleic acid sequence, a nucleic acid sequences of SEQ ID NO: 1, or a variant thereof.

The isolated nucleic acid of the invention which can be used as primers and/or probes can comprise about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 consecutive nucleotides from SEQ ID NO: 1, or sequences complementary SEQ ID NO: 1. The isolated nucleic acid of the invention which can be used as primers and/or probes can comprise from about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to about 20, 25, 30, 35, 40, 45, 50, 55, 60, 65, 70, 75, 80, 85, 90, 95 and 100 consecutive nucleotides from SEQ ID NO: 1, or sequences complementary to SEQ ID NO: 1. The invention is also directed to primer and/or probes which can be labeled by any suitable molecule and/or label known in the art, for example but not limited to fluorescent tags suitable for use in Real Time PCR amplification, for example TaqMan, cybergreen, TAMRA and/or FAM probes; radiolabels, and so forth. In certain embodiments, the oligonucleotide primers and/or probe further comprises a detectable non-isotopic label selected from the group consisting of: a fluorescent molecule, a chemiluminescent molecule, an enzyme, a cofactor, an enzyme substrate, and a hapten.

In certain aspects, the invention is directed to primer sets comprising isolated nucleic acids as described herein, which primer set are suitable for amplification of nucleic acids from samples which comprises canine circoviruses represented SEQ ID NO: 1, or variants thereof. Primer sets can comprise any suitable combination of primers which would allow amplification of a target nucleic acid sequences in a sample which comprises canine circoviruses represented SEQ ID NO: 1, or variants thereof. Amplification can be performed by any suitable method known in the art, for example but not limited to PCR, RT-PCR, transcription mediated amplification (TMA).

Hybridization conditions: As used herein, the phrase “stringent hybridization conditions” refers to conditions under which a probe, primer or oligonucleotide will hybridize to its target sequence, and can hybridize, for example but not limited to, variants of the disclosed polynucleotide sequences, including allelic or splice variants, or sequences that encode orthologs or paralogs of presently disclosed polypeptides. The precise conditions for stringent hybridization are typically sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y (“Sambrook”); and by Haymes et al., “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Numerous variations are possible in the conditions and means by which nucleic acid hybridization can be performed to isolate nucleic sequences having similarity to the nucleic acid sequences known in the art and are not limited to those explicitly disclosed herein. Such an approach may be used to isolate polynucleotide sequences having various degrees of similarity with disclosed nucleic acid sequences, such as, for example, nucleic acid sequences having 60% identity, or about 70% identity, or about 80% or greater identity with disclosed nucleic acid sequences.

Stringent conditions are known to those skilled in the art and can be found in Current Protocols In Molecular Biology, John Wiley & Sons, N.Y. (1989), 6.3.1-7.3.6. In certain embodiments, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× sodium chloride/sodium citrate (SSC), 50 mM Tris-HCl (pH 7.5), 1 nM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. Another non-limiting example of stringent hybridization conditions are hybridization in 6× sodium chloride/sodium citrate (SSC) at about 45° C., followed by one or more washes in 0.2×SSC, 0.1% SDS at 50-65° C. Examples of moderate to low stringency hybridization conditions are well known in the art.

Polynucleotides homologous to the sequences illustrated in SEQ IN NO: 1 and figures can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations.

Encompassed by the invention are polynucleotide sequences that are capable of hybridizing to the claimed polynucleotide sequences, including any of the nucleic acid sequences disclosed herein, and fragments thereof under various conditions of stringency (See, for example, Wahl and Berger (1987) Methods Enzymol. 152: 399-407; and Kimmel (1987) Methods Enzymol. 152: 507-511). With regard to hybridization, conditions that are highly stringent, and means for achieving them, are well known in the art. See, for example, Sambrook et al. (1989) “Molecular Cloning: A Laboratory Manual” (2nd ed. Cold Spring Harbor Laboratory); Berger and Kimmel, eds., (1987) “Guide to Molecular Cloning Techniques”, In Methods in Enzymology: 152: 467-469; and Anderson and Young (1985) “Quantitative Filter Hybridisation.” In: Hames and Higgins, ed., Nucleic Acid Hybridisation. A Practical Approach. Oxford, IRL Press, 73-111.

Stability of DNA duplexes is affected by such factors as base composition, length, and degree of base pair mismatch. Hybridization conditions may be adjusted to allow DNAs of different sequence relatedness to hybridize. The melting temperature (Tm) is defined as the temperature when 50% of the duplex molecules have dissociated into their constituent single strands. The melting temperature of a perfectly matched duplex, where the hybridization buffer contains formamide as a denaturing agent, may be estimated by the following equation: DNA-DNA: Tm(° C.)=81.5+16.6(log [Na+])+0.41(% G+C)−0.62(% formamide)−500/L (1) DNA-RNA: Tm(° C.)=79.8+18.5(log [Na+])+0.58(% G+C)+0.12(% G+C).sup.2−0.5 (% formamide)−820/L (2) RNA-RNA: Tm(C)=79.8+18.5(log [Na+])+0.58(% G+C)+0.12(% G+C).sup.2−0.35 (% formamide)−820/L (3), where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments. The stringency can be adjusted either during the hybridization step or in the post-hybridization washes. Salt concentration, formamide concentration, hybridization temperature and probe lengths are variables that can be used to alter stringency. As a general guidelines high stringency is typically performed at Tm −5° C. to Tm −20° C. moderate stringency at Tm −20° C. to Tm −35° C. and low stringency at Tm −35° SC to Tm −50° C. for duplex >150 base pairs. Hybridization may be performed at low to moderate stringency (25-50° C. below Tm), followed by post-hybridization washes at increasing stringencies. Maximum rates of hybridization in solution are determined empirically to occur at Tm −25° C. for DNA-DNA duplex and Tm −15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. In certain embodiments, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas in certain embodiments high stringency hybridization may be obtained in the presence of at least about 35% formamide, and in other embodiments in the presence of at least about 50% formamide. In certain embodiments, stringent temperature conditions will ordinarily include temperatures of at least about 30° C., and in other embodiment at least about 37° C., and in other embodiments at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed. In a certain embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide. In another embodiment, hybridization will occur at 42 C in 250 mM NaCl, 25 mM trisodium citrate, 1% SDS, 50% formamide. Useful variations on these conditions will be readily apparent to those skilled in the art.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps can be less than about 30 mM NaCl and 3 mM trisodium citrate, and in certain embodiments less than about 15 mM NaCl and 1.5 mM trisodium citrate. For example, the wash conditions may be under conditions of 0.1×SSC to 2.0×SSC and 0.1% SDS at 50-65° C., with, for example, two steps of 10-30 min. One example of stringent wash conditions includes about 2.0×SSC, 0.1% SDS at 65° C. and washing twice, each wash step being about 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homolog, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art.

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid. It may be desirable to select conditions for a particular assay such that a higher signal to noise ratio, that is, about 15× or more, is obtained. Accordingly, an animal nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay. e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman. Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics). BLAST (National Center for Biomedical Information), GAP. BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

An example of an algorithm that is suitable for determining percent sequence identity and sequence similarity is the FASTA algorithm, which is described in Pearson, W. R. & Lipman, D. J., Proc. Natl. Acad. Sci. U.S.A. 85: 2444, 1988. See also W. R. Pearson, Methods Enzymol. 266: 227-258, 1996. Exemplary parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another example of algorithm that is suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:402-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://wwwncbi.nlm.nih.gov/). The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5. N=−4, and a comparison of both strands.

The BLAST algorithm also performs a statistical analysis of the similarity between two sequences (see, e.g., Karlin & Altschul, Proc. Natl. Acad. Sci. U.S.A. 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, and less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of an algorithm that is suitable for multiple DNA and amino acid sequence alignments is the CLUSTALW program (Thompson, J. D. et al., Nucl. Acids. Res. 22:4672-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89:10915-10919, 1992).

“Percent identity” in the context of two or more nucleic acids or polypeptide sequences, refers to the percentage of nucleotides or amino acids that two or more sequences or subsequences contain which are the same. A specified percentage of amino acid residues or nucleotides can be referred to such as: 60% identity, 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 98%, at least 99% or higher nucleotide or amino acid residue identity, when compared and aligned for maximum correspondence, as measured using one of the following sequence comparison algorithms or by visual inspection.

In other aspects, the invention is directed to expression constructs, for example but not limited to plasmids and vectors which comprise the nucleic acid sequence of SEQ ID NO: 1, complementary sequences thereof, and/or variants thereof. Such expression constructs can be prepared by any suitable method known in the art. Such expression constructs are suitable for viral nucleic acid and/or protein expression and purification.

In certain aspects, the invention is directed to iRNA molecules which target nucleic acids from canine circovirus, for example but not limited to SEQ ID NO: 1, and variants thereof, and silence a target gene.

An “iRNA agent” (abbreviation for “interfering RNA agent”) as used herein, is an RNA agent, which can down-regulate the expression of a target gene, e.g. a canine circovirus gene. An iRNA agent may act by one or more of a number of mechanisms, including post-transcriptional cleavage of a target mRNA sometimes referred to in the art as RNAi, or pre-transcriptional or pre-translational mechanisms. An iRNA agent can be a double stranded (ds) iRNA agent.

A “ds iRNA agent” (abbreviation for “double stranded iRNA agent”), as used herein, is an iRNA agent which includes more than one, and in certain embodiments two, strands in which interchain hybridization can form a region of duplex structure. A “strand” herein refers to a contiguous sequence of nucleotides (including non-naturally occurring or modified nucleotides). The two or more strands may be, or each form a part of, separate molecules, or they may be covalently interconnected. e.g. by a linker. e.g. a polyethyleneglycol linker, to form but one molecule. At least one strand can include a region which is sufficiently complementary to a target RNA. Such strand is termed the “antisense strand”. A second strand comprised in the dsRNA agent which comprises a region complementary to the antisense strand is termed the “sense strand”. However, a ds iRNA agent can also be formed from a single RNA molecule which is, at least partly; self-complementary, forming, e.g., a hairpin or panhandle structure, including a duplex region. In such case, the term “strand” refers to one of the regions of the RNA molecule that is complementary to another region of the same RNA molecule.

iRNA agents as described herein, including ds iRNA agents and siRNA agents, can mediate silencing of a gene, e.g., by RNA degradation. For convenience, such RNA is also referred to herein as the RNA to be silenced. Such a gene is also referred to as a target gene. In certain embodiments, the RNA to be silenced is a gene product of a canine circovirus gene.

As used herein, the phrase “mediates RNAi” refers to the ability of an agent to silence, in a sequence specific manner, a target gene. “Silencing a target gene” means the process whereby a cell containing and/or secreting a certain product of the target gene when not in contact with the agent, will contain and/or secret at least 10%, 20%, 30%, 40%, 50%, 60%, 70%, 80%, or 90% less of such gene product when contacted with the agent, as compared to a similar cell which has not been contacted with the agent. Such product of the target gene can, for example, be a messenger RNA (mRNA), a protein, or a regulatory element.

In the anti viral uses of the present invention, silencing of a target gene can result in a reduction in “viral titer” in the cell or in the animal, wherein “reduction in viral titer” refers to a decrease in the number of viable virus produced by a cell or found in an organism undergoing the silencing of a viral target gene. Reduction in the cellular amount of virus produced can lead to a decrease in the amount of measurable virus produced in the tissues of an animal undergoing treatment and a reduction in the severity of the symptoms of the viral infection. iRNA agents of the present invention are also referred to as “antiviral iRNA agents”.

As used herein, a “canine circovirus gene” refers to any one of the genes identified in the canine circovirus genome.

In other aspects, the invention provides methods for reducing viral titer in an animal, by administering to an animal, at least one iRNA which inhibits the expression of a canine circovirus gene.

In other aspects, the invention provides methods for identifying and/or generating anti-viral drugs. For example, in one aspect the invention provides methods for identifying drugs that bind to and/or inhibit the function of the canine circovirus-encoded proteins of the invention, or that inhibit the replication or pathogenicity of the canine circovirus of the invention. Methods of identifying drugs that affect or inhibit a particular drug target, such as high throughput drug screening methods, are well known in the art and can readily be applied to the proteins and viruses of the present invention.

### Isolated Polypeptides

The invention is also directed to isolated polypeptides and variants and derivatives thereof. These polypeptides may be useful for multiple applications, including, but not limited to, generation of antibodies and generation of immunogenic compositions. For example, the invention is directed to any isolated polypeptide encoded by the nucleic sequence acid of SEQ ID NO: 1. A peptide of at least 8 amino acid residues in length can be recognized by an antibody (MacKenzie et al., (1984) Biochemistry 23, 6544-6549. In certain embodiments, the invention is directed to fragments of the polypeptides described herein, that can, for example, be used to generate antibodies.

In one aspect, the invention is directed to polypeptide variants of any isolated polypeptide encoded by the nucleic sequence acid of SEQ ID NO: 1. Variants of any one of the isolated polypeptides encoded by the nucleic sequence acid of SEQ ID NO: 1 include, but are not limited to, polypeptide sequences having at least from about 50% to about 55% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 55.1% to about 60% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 60.1% to about 65% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 65.1% to about 70% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide having at least from about 70.1% to about 75% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 75.1% to about 80% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 80.1% to about 85% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 85.1% to about 90% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 90.1% to about 95% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 95.1% to about 97% identity to that of any isolated polypeptide encoded by the nucleic sequence acid of SEQ ID NO: 1. Variants of any isolated polypeptide encoded by the nucleic sequence acid of SEQ ID NO: 1 include, but are not limited to, polypeptide sequences having at least from about 97.1% to about 99% identity to that of any isolated polypeptide encoded by the nucleic sequence acid of SEQ ID NO: 1.

The invention is directed to a polypeptide sequence comprising from about 10 to about 50 consecutive amino acids from any isolated polypeptide encoded by the nucleic sequence acid of SEQ ID NO: 1. The invention is directed to a polypeptide sequence comprising from about 10 to about 100 consecutive amino acids from any isolated polypeptide encoded by the nucleic sequence acid of SEQ ID NO: 1. The invention is directed to a polypeptide sequence comprising from about 10 to about 150 consecutive amino acids from any isolated polypeptide encoded by the nucleic sequence acid of SEQ ID NO: 1. The invention is directed to a polypeptide sequence comprising from about 10 to about 200 consecutive amino acids from any isolated polypeptide encoded by the nucleic sequence acid of SEQ ID NO: 1. The invention is directed to a polypeptide sequence comprising from about 10 to about 250 consecutive amino acids from any isolated polypeptide encoded by the nucleic sequence acid of SEQ ID NO: 1. The invention is directed to a polypeptide sequence comprising from about 10 to about 300 consecutive amino acids from any isolated polypeptide encoded by the nucleic sequence acid of SEQ ID NO: 1. The invention is directed to a polypeptide sequence comprising from about 10 to about 350 consecutive amino acids from any isolated polypeptide encoded by the nucleic sequence acid of SEQ ID NO: 1. The invention is directed to a polypeptide sequence comprising from about 10 to about 400 consecutive amino acids from any isolated polypeptide encoded by the nucleic sequence acid of SEQ ID NO: 1. The invention is directed to a polypeptide sequence comprising from about 10 to about 450 consecutive amino acids from any isolated polypeptide encoded by the nucleic sequence acid of SEQ ID NO: 1. The invention is directed to a polypeptide sequence comprising from about 10 to about 460 consecutive amino acids from any isolated polypeptide encoded by the nucleic sequence acid of SEQ ID NO: 1. The invention is directed to a polypeptide sequence comprising from about 10 to about 470 consecutive amino acids from any isolated polypeptide encoded by the nucleic sequence acid of SEQ ID NO: 1. The invention is directed to a polypeptide sequence comprising from about 10 to about 480 consecutive amino acids from any isolated polypeptide encoded by the nucleic sequence acid of SEQ ID NO: 1. The invention is directed to a polypeptide sequence comprising from about 10 to about 490 consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 490 consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 550 consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 600 consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 650 consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 650 or more consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 700 or more consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 800 or more consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 900 or more consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 1000 or more consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 1250 or more consecutive amino acids from any isolated polypeptide encoded by the nucleic sequence acid of SEQ ID NO: 1. The invention is further directed to polypeptide sequences having from about 50% to about 99% identity to a polypeptide sequence comprising from about 10 to about 1500 or more consecutive amino acids from any isolated polypeptide encoded by the nucleic sequence acid of SEQ ID NO: 1. In certain embodiments, the invention is directed to isolated and purified peptides.

In certain embodiments, the polypeptides of the present invention can be suitable for use as antigens to detect antibodies against canine circovirus represented by SEQ ID Nos: 1, and variants thereof. In other embodiments, the polypeptides of the present invention which comprise antigenic determinants can be used in various immunoassays to identify animals exposed to and/or samples which comprise canine circovirus represented by SEQ ID NO: 1, and variants thereof.

In another aspect, the invention is directed to an antibody which specifically binds to amino acids from the polypeptide of any isolated polypeptide encoded by the nucleic sequence acid of SEQ ID NO: 1. In one embodiment the antibody is purified. The antibodies can be polyclonal or monoclonal. The antibodies can also be chimeric (i.e., a combination of sequences from more than one species, for example, a chimeric mouse-human immunoglobulin), humanized or fully-human. Species specific antibodies avoid certain of the problems associated with antibodies that possess variable and/or constant regions form other species. The presence of such protein sequences form other species can lead to the rapid clearance of the antibodies or can lead to the generation of an immune response against the antibody by an antibody.

Antibodies can bind to other molecules (antigens) via heavy and light chain variable domains, V_{H} and V_{L}, respectively. The antibodies described herein include, but are not limited to IgY, IgY(ΔFc)), IgG, IgD, IgA, IgM, IgE, and IgL. The antibodies may be intact immunoglobulin molecules, two full length heavy chains linked by disulfide bonds to two full length light chains, as well as subsequences (i.e. fragments) of immunoglobulin molecules, with our without constant region, that bind to an epitope of an antigen, or subsequences thereof (i.e. fragments) of immunoglobulin molecules, with or without constant region, that bind to an epitope of an antigen. Antibodies may comprise full length heavy and light chain variable domains, V_{H} and V_{L}, individually or in any combination.

The basic immunoglobulin (antibody) structural unit can comprise a tetramer. Each tetramer can be composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (Vₗ) and variable heavy chain (V_{H}) refer to these light and heavy chains respectively.

Antibodies may exist as intact immunoglobulins or as a number of well characterized fragments produced by digestion with various peptidases. In particular, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially an Fab with part of the hinge region (see, Fundamental Immunology, W. E. Paul, ed., Raven Press, N.Y. (1993) for more antibody fragment terminology). While the Fab′ domain is defined in terms of the digestion of an intact antibody, one of skill will appreciate that such Fab′ fragments may be synthesized de novo either chemically or by utilizing recombinant DNA methodology.

The Fab′ regions may be derived from antibodies of animal or human origin or may be chimeric (Morrison et al., Proc Natl. Acad. Sci. USA 81, 6851-7855 (1984) both incorporated by reference herein) or humanized (Jones et al., Nature 321, 522-525 (1986), and published UK patent application No. 8707252, both incorporated by reference herein).

An antibody described in this application can include or be derived from any mammal, such as but not limited to, a bird, a dog, a human, a mouse, a rabbit, a rat, a rodent, a primate, or any combination thereof and includes isolated avian, human, primate, rodent, mammalian, chimeric, humanized and/or CDR-grafted or CDR-adapted antibodies, immunoglobulins, cleavage products and other portions and variants thereof.

Any method for producing antibodies can be used to generate the antibodies described herein. Exemplary methods include animal inoculation, phage display, transgenic mouse technology and hybridoma technology.

Methods for generating avain antibodies can also be used to generate the antibodies described herein. In avaians, the egg yolk can be used an antibody source (Altchul et al., Nature Genetics, 1994, 6:119-129). For a review of preimmune diversification and antibody generation in avians, see Reynaud et al., Cell 40, 283-291, 1985 and Thompson et al., Cell 48, 369-378, 1987. In birds, the bursa of Fabricius is the site where B cells undergo gene conversion and are selected for the ability to produce antibodies to antigens. Unlike mammals, the generation of antibody binding specities occurs before hatching rather than throughout their lives. Another difference between avians and mammals is that the major immunoglobulin is IgY rather than IgG. A small version of IgY lacking a full Fc region (IgY(ΔFc)) is also known to be produced in avians. (Zimmerman, et al, (1971) Biochemistry 10: 482-488).

Any methods for producing antibodies in animals can be used to produce the antibodies described herein.

Antibodies useful in the embodiments of the invention can be derived in several ways well known in the art. In one aspect, the antibodies can be obtained using any of the techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2.sup.nd Edition, Cold Spring Harbor, N.Y. (1989); Harlow and Lane, Antibodies, A Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY. N.Y. (1997-2001).

Methods for purifying IgY form egg yolk sacs are also known in the art. See, for example, Polson et al. Immunol Invest. 1985 August: 14(4):323-7; Akita and Nakai, Immunol Methods. 1993 Apr. 2; 160(2):207-14; Akita and Nakai, J Immunol Methods. 1993 Jun. 18; 162(2):155-64 and U.S. Pat. Nos. 4,357,272, 4,550,019, 5,080,895, 5,420,253 and 5,367,054.

The antibodies may also be obtained from selecting from libraries of such domains or components, e.g. a phage library. A phage library can be created by inserting a library of random oligonucleotides or a library of polynucleotides containing sequences of interest, such as from the B-cells of an immunized animal or human (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol Today 21(8) 371-7). The diversity of a phagemid library can be manipulated to increase and/or alter the immunospecificities of the monoclonal antibodies of the library to produce and subsequently identify additional, desirable, human monoclonal antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more human framework sequences and introducing collections of CDR cassettes derived from human antibody repertoires or through designed variation (Kretzschmar and von Ruden 2000, Current Opinion in Biotechnology, 13:598-602). The positions of diversity are not limited to CDRs but can also include the framework segments of the variable regions or may include other than antibody variable regions, such as peptides.

Other target binding components which may include other than antibody variable regions are ribosome display, yeast display, and bacterial displays. Ribosome display is a method of translating mRNAs into their cognate proteins while keeping the protein attached to the RNA. The nucleic acid coding sequence is recovered by RT-PCR (Mattheakis, L. C. et al. 1994. Proc Natl Acad Sci USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based fusion of the target to exported bacterial proteins that associate with the cell membrane or cell wall (Chen and Georgiou 2002. Biotechnol Bioeng, 79:496-503).

In comparison to hybridoma technology, phage and other antibody display methods afford the opportunity to manipulate selection against the antigen target in vitro and without the limitation of the possibility of host effects on the antigen or vice versa.

Specific examples of antibody subsequences include, for example, Fab, Fab′, (Fab′)₂, Fv, or single chain antibody (SCA) fragment (e.g., scFv). Subsequences include portions which retain at least part of the function or activity of full length sequence. For example, an antibody subsequence will retain the ability to selectively bind to an antigen even though the binding affinity of the subsequence may be greater or less than the binding affinity of the full length antibody.

Pepsin or papain digestion of whole antibodies can be used to generate antibody fragments. In particular, an Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An (Fab′)₂ fragment of an antibody can be obtained by treating a whole antibody molecule with the enzyme pepsin, without subsequent reduction. An Fab′ fragment of an antibody molecule can be obtained from (Fab′)₂ by reduction with a thiol reducing agent, which yields a molecule consisting of an intact light chain and a portion of a heavy chain. Two Fab′ fragments are obtained per antibody molecule treated in this manner.

An Fv fragment is a fragment containing the variable region of a light chain V_{L} and the variable region of a heavy chain V_{H} expressed as two chains. The association may be non-covalent or may be covalent, such as a chemical cross-linking agent or an intermolecular disulfide bond (Inbar et al., (1972) Proc. Natl. Acad. Sci. USA 69:2659; Sandhu (1992) Crit. Rev. Biotech. 12:437).

A single chain antibody (“SCA”) is a genetically engineered or enzymatically digested antibody containing the variable region of a light chain V_{L} and the variable region of a heavy chain, optionally linked by a flexible linker, such as a polypeptide sequence, in either V_{L}-linker-V_{H} orientation or in V_{H}-linker-V_{L} orientation. Alternatively, a single chain Fv fragment can be produced by linking two variable domains via a disulfide linkage between two cysteine residues. Methods for producing scFv antibodies are described, for example, by Whitlow et al. (1991) In: Methods: A Companion to Methods in Enzymology 2:97; U.S. Pat. No. 4,946,778; and Pack et al., (1993) Bio/Technology 11:1271.

Other methods of producing antibody subsequences, such as separation of heavy chains to form monovalent light-heavy chain fragments, further cleavage of fragments, or other enzymatic, chemical, or genetic techniques may also be used, provided that the subsequences bind to the antigen to which the intact antibody binds.

Antibodies used in the invention, include full length antibodies, subsequences (e.g., single chain forms), dimers, trimers, tetramers, pentamers, hexamers or any other higher order oligomer that retains at least a part of antigen binding activity of monomer. Multimers can comprise heteromeric or homomeric combinations of full length antibody, subsequences, unmodified or modified as set forth herein and known in the art. Antibody multimers are useful for increasing antigen avidity in comparison to monomer due to the multimer having multiple antigen binding sites. Antibody multimers are also useful for producing oligomeric (e.g., dimer, trimer, tertamer, etc.) combinations of different antibodies thereby producing compositions of antibodies that are multifunctional (e.g., bifunctional, trifunctional, tetrafunctional, etc.).

Antibodies can be produced through chemical crosslinking of the selected molecules (which have been produced by synthetic means or by expression of nucleic acid that encode the polypeptides) or through recombinant DNA technology combined with in vitro, or cellular expression of the polypeptide, and subsequent oligomerization. Antibodies can be similarly produced through recombinant technology and expression, fusion of hybridomas that produce antibodies with different epitopic specificities, or expression of multiple nucleic acid encoding antibody variable chains with different epitopic specificities in a single cell.

Antibodies may be either joined directly or indirectly through covalent or non-covalent binding, e.g. via a multimerization domain, to produce multimers. A “multimerization domain” mediates non-covalent protein-protein interactions. Specific examples include coiled-coil (e.g., leucine zipper structures) and alpha-helical protein sequences. Sequences that mediate protein-protein binding via Van der Waals' forces, hydrogen bonding or charge-charge bonds are also can also be used as multimerization domains. Additional examples include basic-helix-loop-helix domains and other protein sequences that mediate heteromeric or homomeric protein-protein interactions among nucleic acid binding proteins (e.g., DNA binding transcription factors, such as TAFs). One specific example of a multimerization domain is p53 residues 319 to 360 which mediate tetramer formation. Another example is human platelet factor 4, which self-assembles into tetramers. Yet another example is extracellular protein TSP4, a member of the thrombospondin family, which can form pentamers. Additional specific examples are the leucine zippers of jun, fos, and yeast protein GCN4.

Antibodies may be directly linked to each other via a chemical cross linking agent or can be connected via a linker sequence (e.g., a peptide sequence) to form multimers.

The antibodies of the present invention can be used to modulate the activity of any polypeptide encoded by the nucleic sequence acid of SEQ ID NO: 1, variants or fragments thereof. In certain aspects, the invention is directed to a method for treating an animal (e.g. a dog), the method comprising administering to the animal an antibody which specifically binds to amino acids from the polypeptide of any polypeptide encoded by the nucleic sequence acid of SEQ ID NO: 1. In certain embodiments, antibody binding to the polypeptide of any polypeptide encoded by the nucleic sequence acid of SEQ ID NO: 1 may interfere or inhibit the function of the polypeptide, thus providing a method to inhibit virus propagation and spreading.

In other embodiments, the antibodies of the invention can be used to purify polypeptides of any polypeptide encoded by the nucleic sequence acid of SEQ ID NO: 1, variants or fragments thereof. In other embodiments, the antibodies of the invention can be used to identify expression and localization of the polypeptide of any polypeptide encoded by the nucleic sequence acid of SEQ ID NO: 1, variants, fragments or domains thereof. Analysis of expression and localization of the polypeptide of any polypeptide encoded by the nucleic sequence acid of SEQ ID NO: 1 can be useful in determining potential role of the polypeptide of any polypeptide encoded by the nucleic sequence acid of SEQ ID NO: 1.

In other embodiments, the antibodies of the present invention can be used in various immunoassays to identify animals exposed to and/or samples which comprise antigens from canine circovirus represented by SEQ ID Nos: 1, and variants thereof.

Any suitable immunoassay which can lead to formation of antigen-antibody complex can also be used. Variations and different formats of immunoassays, for example but not limited to ELISA, lateral flow assays for detection of analytes in samples, immunoprecipitation, are known in the art. In various embodiments, the antigen and/or the antibody can be labeled by any suitable label or method known in the art. For example enzymatic immunoassays may use solid supports, or immunoprecipitation. Immunoassays which amplify the signal from the antigen-antibody immune complex can also be used with the methods described herein.

In certain aspects the invention provides methods for assaying a sample to determine the presence or absence of a canine circovirus comprising SEQ ID NOs: 1, as provided by the invention, and variants thereof. In certain embodiments, methods for assaying a sample, include, but are not limited to, methods which can detect the presence of nucleic acids, methods which can detect the presence of antigens, methods which can detect the presence of antibodies against antigens from polypeptides encoded by SEQ ID NO: 1, or any polypeptide encoded by the nucleic sequence acid of SEQ ID NO: 1, as provided by the invention, and variants thereof.

### Immunogenic Compositions

In certain aspects, the present invention provides immunogenic compositions capable of inducing an immune response against canine circovirus including the canine circovirus of the invention comprising any one of SEQ ID NO: 1. In one embodiment, the immunogenic compositions are capable of ameliorating the symptoms of a canine circovirus infection and/or of reducing the duration of a canine respiratory and gastrointestinal disease. In another embodiment, the immunogenic compositions are capable of inducing protective immunity against canine respiratory and gastrointestinal disease. The immunogenic compositions of the invention can be effective against the canine circoviruses disclosed herein, and may also be cross-reactive with, and effective against, multiple different clades and strains of canine circovirus, and against other Circoviridae.

The types of immunogenic composition encompassed by the invention include, but are not limited to, attenuated live viral immunogenic compositions, inactivated (killed) viral immunogenic compositions, and subunit immunogenic compositions.

The canine circoviruses of the invention may be attenuated by removal or disruption of those viral sequences whose products cause or contribute to the disease and symptoms associated with canine circovirus infection, and leaving intact those sequences required for viral replication. In this way an attenuated canine circoviruscan be produced that replicates in animals, and induces an immune response in animals, but which does not induce the deleterious disease and symptoms usually associated with canine circovirus infection. One of skill in the art can determine which canine circovirus sequences can or should be removed or disrupted, and which sequences should be left intact, in order to generate an attenuated canine circovirus suitable for use as an immunogenic composition.

The novel canine circovirus of the invention may be also be inactivated, such as by chemical treatment, to “kill” the viruses such that they are no longer capable of replicating or causing disease in animals, but still induce an immune response in an animal (e.g. a dog). There are many suitable viral inactivation methods known in the art and one of skill in the art can readily select a suitable method and produce an inactivated “killed” canine circovirus suitable for use as an immunogenic composition.

The immunogenic compositions of the invention may comprise subunit immunogenic compositions. Subunit immunogenic compositions include nucleic acid immunogenic compositions such as DNA immunogenic compositions, which contain nucleic acids that encode one or more viral proteins or subunits, or portions of those proteins or subunits. When using such immunogenic compositions, the nucleic acid is administered to the animal, and the immunogenic proteins or peptides encoded by the nucleic acid are expressed in the animal, such that an immune response against the proteins or peptides is generated in the animal. Subunit immunogenic compositions may also be proteinaceous immunogenic compositions, which contain the viral proteins or subunits themselves, or portions of those proteins or subunits.

To make the nucleic acid and DNA immunogenic compositions of the invention the canine circovirus sequences disclosed herein may be incorporated into a plasmid or expression vector containing the nucleic acid that encodes the viral protein or peptide. Any suitable plasmid or expression vector capable of driving expression of the protein or peptide in the animal may be used. Such plasmids and expression vectors should include a suitable promoter for directing transcription of the nucleic acid. The nucleic acid sequence(s) that encodes the canine circovirus protein or peptide may also be incorporated into a suitable recombinant virus for administration to the animal. Examples of suitable viruses include, but are not limited to, vaccinia viruses, retroviruses, adenoviruses and adeno-associated viruses. One of skill in the art could readily select a suitable plasmid, expression vector, or recombinant virus for delivery of the canine circovirus nucleic acid sequences of the invention.

To produce the proteinaceous immunogenic compositions of the invention, the canine circovirus nucleic acid sequences of the invention are delivered to cultured cells, for example by transfecting cultured cells with plasmids or expression vectors containing the canine circovirus nucleic acid sequences, or by infecting cultured cells with recombinant viruses containing the canine circovirus nucleic acid sequences. The canine circovirus proteins or peptides may then be expressed in the cultured cells and purified. The purified proteins can then be incorporated into compositions suitable for administration to animals. Methods and techniques for expression and purification of recombinant proteins are well known in the art, and any such suitable methods may be used.

Subunit immunogenic compositions of the present invention may encode or contain any of the canine circovirus proteins or peptides described herein, or any portions, fragments, derivatives or mutants thereof, that are immunogenic in an animal. One of skill in the art can readily test the immunogenicity of the canine circovirus proteins and peptides described herein, and can select suitable proteins or peptides to use in subunit immunogenic compositions.

The immunogenic compositions of the invention comprise at least one canine circovirus-derived immunogenic component, such as those described herein. The compositions may also comprise one or more additives including, but not limited to, one or more pharmaceutically acceptable carriers, buffers, stabilizers, diluents, preservatives, solubilizers, liposomes or immunomodulatory agents. Suitable immunomodulatory agents include, but are not limited to, adjuvants, cytokines, polynucleotide encoding cytokines, and agents that facilitate cellular uptake of the canine circovirus-derived immunogenic component.

Immunogenic compositions for use in accordance with the present invention thus may be formulated in a conventional manner using one or more physiologically acceptable carriers comprising excipients and auxiliaries which facilitate processing of the active compounds into preparations which can be used to induce an immunogenic response. These immunogenic compositions may be manufactured in a manner that is itself known, e.g. by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein or other active ingredient of the present invention is administered orally, protein or other active ingredient of the present invention can be in the form of a tablet, capsule, powder, solution or elixir. When administered in tablet form, the immunogenic composition of the invention may additionally contain a solid carrier such as a gelatin or an adjuvant. The tablet, capsule, and powder contain from about 5 to 95% protein or other active ingredient of the present invention, and from about 25 to 90% protein or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the immunogenic composition may further contain physiological saline solution, dextrose or other saccharide solution, or glycols such as ethylene glycol, propylene glycol or polyethylene glycol. When administered in liquid form, the immunogenic composition contains from about 0.5 to 90% by weight of protein or other active ingredient of the present invention, and from about 1 to 50% protein or other active ingredient of the present invention.

When a therapeutically effective amount of protein or other active ingredient of the present invention is administered by intravenous, cutaneous or subcutaneous injection, protein or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. One immunogenic composition for intravenous, cutaneous, or subcutaneous injection can contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The immunogenic composition of the present invention may also contain stabilizers, preservatives, buffers, antioxidants, or other additives known to those of skill in the art. For injection, the agents of the invention may be formulated in aqueous solutions, physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with immunogenicly acceptable carriers well known in the art. Such carriers enable the compounds of the invention to be formulated as tablets, pills, dragees, capsules, liquids, gels, syrups, slurries, suspensions and the like, for oral ingestion by a patient to be treated. Immunogenic preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Immunogenic preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Immunogenic formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient maybe in powder form for constitution with a suitable vehicle, e.g., sterile pyrogen-free water, before use.

The compounds may also be formulated in rectal compositions such as suppositories or retention enemas, e.g. containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A carrier for hydrophobic compounds of the invention can be a co-solvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone: and other sugars or polysaccharides may substitute for dextrose. Alternatively, other delivery systems for hydrophobic immunogenic compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The immunogenic compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with immunogenicly compatible counter ions. Such immunogenicly acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The immunogenic composition of the invention may be in the form of a complex of the protein(s) or other active ingredient of present invention along with protein or peptide antigens.

The immunogenic composition of the invention may be in the form of a liposome in which protein of the present invention is combined, in addition to other acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

Other additives that are useful in immunogenic composition formulations are known and will be apparent to those of skill in the art.

An “immunologically effective amount” of the compositions of the invention may be administered to an animal. As used herein, the term “immunologically effective amount” refers to an amount capable of inducing, or enhancing the induction of, the desired immune response in an animal. The desired response may include, inter alia, inducing an antibody or cell-mediated immune response, or both. The desired response may also be induction of an immune response sufficient to ameliorate the symptoms of a canine respiratory and gastrointestinal disease, reduce the duration of a canine respiratory and gastrointestinal disease, and/or provide protective immunity in an animal against subsequent challenge with a canine circovirus. An immunologically effective amount may be an amount that induces actual “protection” against canine respiratory and gastrointestinal disease, meaning the prevention of any of the symptoms or conditions resulting from canine respiratory and gastrointestinal disease in animals. An immunologically effective amount may also be an amount sufficient to delay the onset of symptoms and conditions associated with infection, reduce the degree or rate of infection, reduce in the severity of any disease or symptom resulting from infection, and reduce the viral load of an infected animal.

One of skill in the art can readily determine what is an “immunologically effective amount” of the compositions of the invention without performing any undue experimentation. An effective amount can be determined by conventional means, starting with a low dose of and then increasing the dosage while monitoring the immunological effects. Numerous factors can be taken into consideration when determining an optimal amount to administer, including the size, age, and general condition of the animal, the presence of other drugs in the animal, the virulence of the particular canine circovirus against which the animal is being vaccinated, and the like. The actual dosage is can be chosen after consideration of the results from various animal studies.

The immunologically effective amount of the immunogenic composition may be administered in a single dose, in divided doses, or using a “prime-boost” regimen. The compositions may be administered by any suitable route, including, but not limited to parenteral, intradermal, transdermal, subcutaneous, intramuscular, intravenous, intraperitoneal, intranasal, oral, or intraocular routes, or by a combination of routes. The compositions may also be administered using a “gun” device which fires particles, such as gold particles, onto which compositions of the present invention have been coated, into the skin of an animal. The skilled artisan will be able to formulate the immunogenic composition according to the route chosen.

### Viral Purification

Methods of purification of inactivated virus are known in the art and may include one or more of, for instance gradient centrifugation, ultracentrifugation, continuous-flow ultracentrifugation and chromatography, such as ion exchange chromatography, size exclusion chromatography, and liquid affinity chromatography. Additional method of purification include ultrafiltration and dialfiltration. See J P Gregersen “Herstellung von Virussimpfstoffen aus Zellkulturen” Chapter 4.2 in Pharmazeutische Biotecnology (eds. O. Kayser and R H Mueller) Wissenschaftliche Verlagsgesellschaft, Stuttgart. 2000. See also, O'Neil et al., “Virus Harvesting and Affinity Based Liquid Chromatography. A Method for Virus Concentration and Purification”, Biotechnology (1993) 11:173-177: Prior et al., “Process Development for Manufacture of Inactivated HIV-1”, Pharmaceutical Technology (1995) 30-52; and Majhdi et al., “Isolation and Characterization of a Coronavirus from Elk Calves with diarrhea” Journal of Clinical Microbiology (1995) 35(11): 2937-2942.

Other examples of purification methods suitable for use in the invention include polyethylene glycol or ammonium sulfate precipitation (see Trepanier et al., “Concentration of human respiratory syncytial virus using ammonium sulfate, polyethylene glycol or hollow fiber ultrafiltration” Journal of Virological Methods (1981) 3(4):201-711; Hagen et al., “Optimization of Poly(ethylene glycol) Precipitation of Hepatitis Virus Used to prepare VAQTA, a Highly Purified Inactivated Vaccine” Biotechnology Progress (1996) 12:406-412; and Carlsson et al., “Purification of Infectious Pancreatic Necrosis Virus by Anion Exchange Chromatography Increases the Specific Infectivity” Journal of Virological Methods (1994) 47:27-36) as well as ultrafiltration and microfiltration (see Pay et al., Developments in Biological Standardization (1985) 60:171-174; Tsurumi et al., “Structure and filtration performances of improved cuprammonium regenerated cellulose hollow fibre (improved BMM hollow fibre) for virus removal” Polymer Journal (1990) 22(12):1085-1100; and Makino et al., “Concentration of live retrovirus with a regenerated cellulose hollow fibre, BMM”, Archives of Virology (1994) 139(1-7):87-96.).

Viruses can be purified using chromatography, such as ion exchange, chromatography. Chromatic purification allows for the production of large volumes of virus containing suspension. The viral product of interest can interact with the chromatic medium by a simple adsorption/desorption mechanism, and large volumes of sample can be processed in a single load. Contaminants which do not have affinity for the adsorbent pass through the column. The virus material can then be eluted in concentrated form.

Anion exchange resins that may be used include DEAE, EMD TMAE. Cation exchange resins may comprise a sulfonic acid-modified surface. Viruses can be purified using ion exchange chromatography comprising a strong anion exchange resin (e.g. EMD TMAE) for the first step and EMD-SO.sub.3 (cation exchange resin) for the second step. A metal-binding affinity chromatography step can optionally be included for further purification. (See, e.g., WO 97/06243).

A resin such as Fractogel EMD can also be used This synthetic methacrylate based resin has long, linear polymer chains covalently attached and allows for a large amount of sterically accessible ligands for the binding of biomolecules without any steric hindrance.

Column-based liquid affinity chromatography is another purification method that can be used invention. One example of a resin for use in purification method is Matrex Cellufine Sulfate (MCS). MCS consists of a rigid spherical (approx. 45-105 .mu·m diameter) cellulose matrix of 3,000 Dalton exclusion limit (its pore structure excludes macromolecules), with a low concentration of sulfate ester functionality on the 6-position of cellulose. As the functional ligand (sulfate ester) is relatively highly dispersed, it presents insufficient cationic charge density to allow for most soluble proteins to adsorb onto the bead surface. Therefore the bulk of the protein found in typical virus pools (cell culture supernatants, e.g. pyrogens and most contaminating proteins, as well as nucleic acids and endotoxins) are washed from the column and a degree of purification of the bound virus is achieved.

The rigid, high-strength beads of MCS tend to resist compression. The pressure/flow characteristics the MCS resin permit high linear flow rates allowing high-speed processing, even in large columns, making it an easily scalable unit operation. In addition a chromatographic purification step with MCS provides increased assurance of safety and product sterility, avoiding excessive product handling and safety concerns. As endotoxins do not bind to it, the MCS purification step allows a rapid and contaminant free depyrogenation. Gentle binding and elution conditions provide high capacity and product yield. The MCS resin therefore represents a simple, rapid, effective, and cost-saving means for concentration, purification and depyrogenation. In addition, MCS resins can be reused repeatedly.

Inactivated viruses may be further purified by gradient centrifugation, or density gradient centrifugation. For commercial scale operation a continuous flow sucrose gradient centrifugation would be an option. This method is widely used to purify antiviral immunogenic compositions and is known to one skilled in the art (See J P Gregersen “Herstellung von Virussimpfstoffen aus Zellkulturen” Chapter 4.2 in Pharmazeutische Biotechnology (eds. O. Kayser and R H Mueller) Wissenschaftliche Verlagsgesellschaft, Stuttgart, 2000.)

Additional purification methods which may be used to purify viruses of the invention include the use of a nucleic acid degrading agent, a nucleic acid degrading enzyme, such as a nuclease having DNase and RNase activity, or an endonuclease, such as from Serratia marcescens, membrane adsorbers with anionic functional groups or additional chromatographic steps with anionic functional groups (e.g. DEAE or TMAE). An ultrafiltration/dialfiltration and final sterile filtration step could also be added to the purification method.

The purified viral preparation of the invention is substantially free of contaminating proteins derived from the cells or cell culture and can comprises less than about 1000, 500, 250, 150, 100, or 50 pg cellular nucleic acid/.mu·g virus antigen, and less than about 1000, 500, 250, 150, 100, or 50 pg cellular nucleic acid/dose. The purified viral preparation can also comprises less than about 20 pg or less than about 10 pg. Methods of measuring host cell nucleic acid levels in a viral sample are known in the art. Standardized methods approved or recommended by regulatory authorities such as the WHO or the FDA can be used.

### EXAMPLES

### Example 1

### Circovirus Genetic Analysis

Described herein is the complete genome of a highly divergent Circovirus species in Dogs blood. In certain embodiments, the virus described herein can be a virus that is a pathogen of dogs and cats. The reported novel virus species belongs to family Circoviridae. Described herein is the complete genome sequence, genomic organization, translated protein sequence of this new virus provisionally named Canine Circovirus (CaCV). The phylogenetic analysis performed using nucleotide and protein alignments confirms CaCV as unique and highly divergent to any other known animal circoviruses.

The virus described herein is the first circovirus known to infect and can cause diseases in Dogs and cats (canine and felines host). Phylogenetically CaCV is distantly related to Procine circoviruses, known to cause diseases in pigs. CaCV replicase protein showed <55% protein identity with replicase protein of any known animal circovirus. CaCV capsid protein showed <25% protein identity with capsid protein of any known animal circovirus. CaCV is also the only virus of its kind described to cause infection of dogs and cats. Complete genome sequence of CaCV and its predicted proteins (structural/non-structural proteins). The polypeptides described as being encoded by the genomic CaCV sequences described herein is not intended to be limiting. Other polypeptides encoded by the genomic CaCV sequences described herein are also within the scope of the invention.

The sequences and methods described herein are useful for, inter alia, developing drugs, diagnostics and immunogenic compositions.

### Example 2

### Complete Genome Sequence of the First Canine Circovirus

Highly divergent Circovirus were identified in serum samples of several dogs. The Canine circovirus genotype-1 (CaCV-1) represents the first circovirus known to infect dogs and the only authentic mammalian circovirus, besides Porcine circoviruses. Described herein is the complete genome sequence of the CaCV-1 strain—NY214, which will help towards understanding the evolutionary and pathogenic characteristics of mammalian circoviruses.

Described herein is the discovery of a highly divergent circovirus found in serum samples from several dogs. Phylogenetic analysis indicates that canine circovirus genotype 1 (CaCV-1) represents the first circovirus reported in dogs and is genetically most closely related to the only known mammalian circovirus, porcine circovirus. Here we report the complete genome sequence of the CaCV-1 strain NY214, which will help toward understanding the evolutionary and pathogenic characteristics of mammalian circoviruses.

Circoviruses are genetically diverse nonenveloped viruses with a small monomeric single-strand circular DNA genome and belong to the family Circoviridae, which comprises two genera, Circovirus and Gyrovirus (King et al, 2011). The genus Circovirus includes six recognized species that infect mammals and birds, namely, porcine circovirus 1 (Tischer et al. 1974), porcine circovirus 2 (Meehan et al, 1998), canary circovirus (Todd et al, 2001), goose circovirus (Todd et al., 2010), pigeon circovirus (Todd et al, 2010), and beak and feather disease virus (Ritchie et al, 1989).

Several new circoviruses have been recently identified in animal feces and environmental samples; however the natural hosts of most of these viruses remain unidentified (Delwart 2012. Li et al. 2010, Rosario et al. 2009). Canine circovirus genotype 1 (CaCV-1) was found in serum samples from several dogs (6 of 205 animals tested) and thus represents the first nonporcine circovirus confirmed to infect mammals.

The complete genome of CaCV-1 (strain—NY214) comprises 2063 nt as covalently closed circular DNA with a GC content of 51.7%. TG and GG were the most abundant dinucleotides with observed to expect frequency ratio of >1.34. The genome contains two putative open reading frames (ORF), on complementary strands in opposite orientation, that code for viral raplicase (303 aa) and capsid protein (270 aa). Similar to other animal circoviruses. CaCV has two intergenic non-coding regions that are 135 and 203 nt long, the non-coding region between two major ORF's contains a thermodynamically stable stem loop for initiation of rolling-circle replication and a unique nonanucleotide sequence as “TAGTATTAC” (1) (SEQ ID NO: 5). In CaCV-1 the palindrome sequence at the origin of replication site comprised of 12 nucleotide pairs (stem) and an open loop of 10 nt (CATAGTATTA) (SEQ ID NO: 6).

Similar to other animal circoviruses, the amino terminus of putative CaCV capsid protein contains a 30 aa long arginine (R) rich stretch. The capsid and replicase proteins of CaCV-1 shares <25% and <50% identities respectively, with the known animal circoviruses. Phylogenetic analysis of conserved replicase protein indicates that CaCV 1 is genetically most closely related to Porcine circoviruses. According to the classification criteria by the International Committee on Taxonomy of Viruses (ICTV) (www.ictvdb.org), circoviruses of same species should share >75% and >70% nucleotide identity in their complete genome and capsid protein sequences, respectively. Comparative genetic analysis of CaCV-1 therefore indicates that it be classified as a prototype of new species in the genus Circovirus of family Circoviridae.

The availability of CaCV complete genome sequence will facilitate studies to determine its pathogenic potential in infected animals and for understanding the evolutionary relationship with other pathogenic and non-pathogenic circoviruses that infect other domestic animal species, like pigs. The availability of this sequence will also enable others in the virology community to investigate the epidemiology, evolutionary biology, and pathobiology of mammalian circovirus infection and allow development of molecular reagents that can be used to identify more novel circoviruses that infect other mammalian species.

The 203-nt-long intergenic noncoding region of CaCV-1 has 91% nucleotide identity over 150 nt of sequence with pine marten torque teno virus (van den Brand et al, 2012), providing the first direct evidence of an evolutionary relationship between two distinct virus families that includes genetically diverse viruses with single-stranded DNA (ssDNA) circular genomes, Circoviridae and Anelloviridae.

Nucleotide sequence accession number. The GenBank accession number of the CaCV-1 strain NY214 complete genome sequence is JQ821392.

### REFERENCES

1. Cheung A K. 2012. Porcine circovirus: transcription and DNAreplication. Virus Res. 164:46-53.
2. Delwart E, Li L. 2012. Rapidly expanding genetic diversity and host range of the Circoviridae viral family and other Rep encoding small circular ssDNA genomes. Virus Res. 164:114-121.
3. King A M Q, Lefkowitz E, Adams M J, Carstens E B (ed). 2011. Virus taxonomy: ninth report of the International Committee on Taxonomy of Viruses. Elsevier, Philadelphia, Pa.
4. Li L. et al. 2010. Multiple diverse circoviruses infect farm animals and are commonly found in human and chimpanzee feces. J. Virol. 84:1674-1682.
5. Meehan B M, et al. 1998. Characterization of novel circovirus DNAs associated with wasting syndromes in pigs. J. Gen. Virol. 79:2171-2179.
6. Ritchie B W, Niagro F D, Lukert P D, Steffens W L III, Latimer K S. 1989. Characterization of a new virus from cockatoos with psittacine beak and feather disease. Virology 171:83-88.
7. Rosario K, Duffy S, Breitbart M. 2009. Diverse circovirus-like genome architectures revealed by environmental metagenomics. J. Gen. Virol. 90: 2418-2424.
8. Tischer I. Rasch R. Tochtermann G. 1974. Characterization of papovavirus- and picornavirus-like particles in permanent pig kidney cell lines. Zentralbl. Bakteriol. Orig. A 226:153-167.
9. Todd D, et al. 2001. Nucleotide sequence-based identification of a novel circovirus of canaries. Avian Pathol. 30:321-325.
10. Todd D. Weston J H, Soike D, Smyth J A. 2001. Genome sequence determinations and analyses of novel circoviruses from goose and pigeon. Virology 286:354-362.
11. van den Brand J M, et al. 2012. Metagenomic analysis of the viral flora of pine marten and European badger feces. J. Virol. 86:2360-2365.

### Example 3

### Production of Immunogenic Compositions

The circoviruses and immunogenic compositions described herein can be produced in cells. Production of the circoviruses and immunogenic compositions described herein may also be accomplished on any useful media and permissive cell or tissues, which may be derived from avian or mammalian cell lines derived from human, canine, feline, equine, bovine or porcine cell lines. As used herein, a cell or a tissue can include, but is not limited to individual cells, tissues, organs, insect cells, avian cells, mammalian cells, hybridoma cells, primary cells, continuous cell lines, and/or genetically engineered cells, such as recombinant cells expressing a virus. For example, production of the circoviruses and immunogenic compositions can be in any cell type, including but not limited to mammalian cells. Cell lines suitable for producing the circoviruses and immunogenic compositions described herein include, but are not limited to dog kidney cells, BSC-1 cells, LLC-MK cells, CV-1 cells, CHO cells, COS cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells. MRC-5 cells, T-FLY cells. BHK cells. SP2/0 cells, NS0, PerC6 (human retina cells), chicken embryo cells or derivatives, embryonated egg cells, embryonated chicken eggs or derivatives thereof.

The cell culture system for producing the circoviruses and immunogenic compositions described herein can be a traditional adherent monolayer culture. Alternatively, suspension and microcarrier cell culture systems can also be utilized.

Vessels for producing the circoviruses and immunogenic compositions described herein include, but are not limited to, roller bottles. For example, alternatively, other useful cell culture formats include flasks, stacked modules and stir tanks. For viral production, multiplicity of infection (MOI) can be 0.001-0.1 but can range from 0.0001-2.0. The harvest virus from cell culture can be, but is not limited to, any time between day 2 to 5 post-infection, but can range from day 1 to day 7 post-infection.

Cell culture media formulations to suitable for producing the circoviruses and immunogenic compositions described herein include, but are not limited to, Modified Eagle's media MEM, minimum essential media MEM, Dulbecco's modified Eagle's media D-MEM. D-MEM-F12 media, William's E media, RPMI media and analogues and derivative thereof. These can also be specialty cell cultivation and virus growth media as VP-SFM, OptiPro™ SFM, AIM V® media, HyQ SFM4 MegaVir™, EX-CELL™ Vero SFM, EPISERF, ProVero, any 293 or CHO media and analogues and derivatives thereof. The culture media described herein can be supplemented by any additive known from prior art that is applicable for cell and virus cultivation as for example animal sera and fractions or analogues thereof, amino acids, growth factors, hormones, buffers, trace elements, trypsin, sodium pyruvate, vitamins, L-glutamine and biological buffers. Preferable medium is OptiPRO™ SFM supplemented with L-glutamine and trypsin. In certain embodiments, the cell culture media can be supplemented with 0.1 to 10 units of trypsin. Alternatively, plant derived equivalents of trypsin (e.g. Accutase) ranging from 2-100 units can also be used in cell culture. Cell culture media can be used in the absence or presence of animal-derived components. An example of supplementation with an animal-derived component is gamma-irradiated serum ranging from 0.5-10%6 final concentration.

Growth or production of the circoviruses and immunogenic compositions in can also be performed in eggs. For example, circovirus propagation can be accomplished by inoculating embryonated eggs. In certain embodiments, 0-12 day old embryonated eggs can be used for circovirus propagation. In certain embodiments, 7-8 day old embryonated eggs can be used for virus growth. The circovirus can be inoculated into the amniotic cavity of the egg. In certain embodiments, the circovirus will replicate in the cells of the amniotic membrane and large quantities are released back into the amniotic fluid. In certain embodiments, circovirus in the amniotic fluid can be harvested after 2-3 days post inoculation.

Production of the circoviruses and immunogenic compositions in can also be performed using a recombinant expression system that expresses the circovirus, a circoviral protein, a fragment of a bocoviral protein or a variant of a circoviral protein. The expression system can comprise any suitable plasmid or a linear expression construct known in the art.

### Example 4

### Virus Preparation, Attenuation and Inactivation

The immunogenic compositions described herein can comprise an inactivated or killed circovirus vaccine. Inactivated immunogenic composition can made by methods well known in the art. For example, once the circovirus is propagated to high titers, the circovirus antigenic mass could be obtained by methods well known in the art. For example, the circoviral antigenic mass may be obtained by dilution, concentration, or extraction. All of these methods have been employed to obtain appropriate circoviral antigenic mass to produce immunogenic compositions. The circovirus may be inactivated by treatment with formalin (e.g. 0.1-10%), betapropriolactone (BPL) (e.g. 0.01-10%), or with binary ethyleneimine (BEI) (e.g. 1-10 mM), or using other methods known to those skilled in the art.

In addition to killed circovirus production, various means of attenuation are also possible and are well known and described in the art. Attenuation leading to modified live immunogenic compositions can also be used in conjunction with the compositions and methods described herein. Methods of attenuation suitable for use with the viruses described herein include continuous passaging in cell culture, continuous passaging in animals, various methods for generating genetic modifications and ultraviolet or chemical mutagenesis.

Attenuation of circovirus may be achieved through cold-adaptation of an circovirus strain. Cold-adapted circovirus virus strains may be produced by methods which includes passaging a wild-type circovirus virus, followed by selection for circovirus that grows at a reduced temperature. Cold-adapted circovirus can be produced, for example, by sequentially passaging a wild-type circovirus in embryonated cells or chicken eggs at progressively lower temperatures, thereby selecting for certain members of the circovirus mixture which stably replicate at the reduced temperature. A cold-adapted circovirus strain may exhibit a temperature sensitive phenotype. A temperature sensitive cold-adapted circovirus replicates at reduced temperatures, but no longer replicates at certain higher growth temperatures at which the wild-type circovirus will replicate. A temperature at which a temperature sensitive circovirus will grow is referred to herein as a “permissive” temperature for that temperature sensitive circovirus, and a higher temperature at which the temperature sensitive circovirus will not grow, but at which a corresponding wild-type circovirus will grow, is referred to herein as a “non-permissive” temperature for that temperature sensitive circovirus. A cold-adapted circovirus may also be produced through recombinant means. In this approach, one or more specific mutations, associated with identified cold-adaptation, attenuation, temperature sensitivity, or dominant interference phenotypes, can be identified and are introduced back into a wild-type circovirus strain using a reverse genetics approach. Reverse genetics entails can be performed using RNA polymerase complexes isolated from circovirus-infected cells to transcribe artificial circovirus genome segments containing the mutation(s), incorporating the synthesized RNA segment(s) into virus particles using a helper virus, and then selecting for viruses containing the desired changes.

Attenuation of an circovirus may be achieved by serial passaging of a wild-type circovirus strain in cell culture. The circovirus strain can be passaged in a variety of cell systems until its ability to produce disease is lost whilst its immunogenic character is fully retained. Once inoculated into the host, the circovirus may be capable of multiplication to some extent. For example, attenuated circovirus compositions can be prepared from cell line that has been attenuated by serial passage including serial passage at sub-optimal temperatures to a state where it is no longer capable of causing disease, but still capable of eliciting a protective immune response.

Suitable attenuated circovirus strains may also be obtained by serial passaging to obtain an over-attenuated strain. The “over-attenuation” means that the number of passages for attenuation has been substantially greater than what is normally necessary for the removal of pathogenicity. The attenuated circovirus retains its antigenicity after these numerous passages so that its immunogenic ability is not impaired. Such strains produce practically no symptoms or side effects when administered, and thus are safe and efficacious vaccines.

### Example 5

### Immunogenic Composition Dosages

Dose sizes of the immunogenic compositions described herein can be in the range of about 2.0 to 0.1 ml depending on the route of administration, but dose sizes are not limited to this range. For inactivated circovirus compositions can contain suitable TCID50 levels of virus prior to inactivation. One of skill in the art will readily be capable of determining a suitable TCID50 level for the immunogenic compositions described herein. The antigen content in the circovirus preparation can have, but is not limited to, a titer of between 10 to 10,000 units/ml as the amount administered per dose. One of skill in the art will readily be capable of determining a suitable antigen content for the immunogenic compositions described herein.

For immunogenic compositions containing modified live circoviruses or attenuated circoviruses, a therapeutically effective dose can be determined by one of skill in the art. For immunogenic compositions containing circovirus subunit antigens, a therapeutically effective dose can be determined by one of skill in the art. While the amounts and concentrations of adjuvants and additives useful in the context of the present invention can readily be determined by the skilled artisan.

### Example 6

### Administration of Immunogenic Compositions

An animal, for example a dog, can be inoculated with the immunogenic compositions or formulations described herein to generate an immune response. In certain embodiments, inoculation can be performed on an animal (e.g. a dog) that is at least 6 weeks or older. In certain embodiments, the animal (e.g. dog) can receive one or more dosages. In certain embodiments, two or more dosages can be administered to the animal (e.g. dog) 3-4 weeks apart. In certain embodiments, the administration can be by subcutaneous injection. Intramuscular, intradermal, oral, oronasal or nasal routes of administration can also be used to administer the immunogenic compositions or formulations described herein.

What is claimed is:

1. An isolated nucleic acid having the sequence of SEQ ID NO: 1.

2. An isolated nucleic acid having at least about 60% sequence identity to SEQ ID NO: 1.

3. An isolated nucleic acid which comprises at least 10 consecutive nucleotides of SEQ ID NO: 1.

4. An isolated nucleic acid which comprises at least 10 consecutive nucleotides of a sequence having at least about 60% identity to SEQ ID NO: 1.

5. An isolated nucleic acid which comprises consecutive nucleotides having a sequence complementary to the nucleic acid of claim 3 or 4.

6. An isolated polypeptide having the sequence of any of SEQ ID NOs: 2-4.

7. An isolated polypeptide having at least about 80% sequence identity to any of SEQ ID NO: 2-4.

8. An isolated polypeptide comprising at least 8 consecutive amino acids of any of SEQ ID NOs 2-4.

9. An isolated polypeptide comprising at least 8 amino acids having at least about 80% identity to the sequence of any of SEQ ID NOs 2-4.

10. An isolated nucleic acid encoding the polypeptide of any of claims 6-9.

11. An isolated antibody that specifically binds to a polypeptide of any of claims 6-9.

12. An immunogenic composition comprising at least about 24 consecutive nucleotides from the nucleic acid of claim 1 or 2.

13. An immunogenic composition comprising at least about 8 consecutive amino acids of claim 6 or 7.

14. A method of inducing an immune response in an animal, the method comprising administering the immunogenic composition of claim 12 or 13.

15. A synthetic nucleic acid comprising at least about 10 nucleotides of the isolated nucleic acid of claim 1 or 2.

16. A synthetic nucleic acid comprising at least about 10 nucleotides complementary to the isolated nucleic acid of claim 1 or 2.

17. A method for determining the presence or absence of canine circovirus a biological sample, the method comprising:
a) contacting nucleic acid from a biological sample with at least one primer which is a synthetic nucleic acid of claim 15 or 16,
b) subjecting the nucleic acid and the primer to amplification conditions, and
c) determining the presence or absence of amplification product, wherein the presence of amplification product indicates the presence of RNA associated with of canine circovirus the sample.

18. A primer set for determining the presence or absence of canine circovirus a biological sample, wherein the primer set comprises at least one synthetic nucleic acid sequence selected from the group consisting of:
a) a synthetic nucleic acid of claim 15, and
b) a synthetic nucleic acid of claim 16.

19. A method for determining whether or not a sample contains of canine circovirus, the method comprising:
a) contacting a biological sample with an antibody that specifically binds to a polypeptide of any of claims 6-9, and
b) determining whether or not the antibody binds to an antigen in the biological sample, wherein binding indicates that the biological sample contains canine circovirus.

20. The method of claim 19, wherein the determining comprises use of a lateral flow assay or ELISA.

21. A method for determining whether or not a biological sample has been infected by canine circovirus, the method comprising:
a) determining whether or not a biological sample contains antibodies that specifically bind to a polypeptide of claim 6 or 7.

22. The method of claim 21, wherein the determining comprises determining whether the antibodies are IgY antibodies, wherein detection of IgY antibodies is indicative of a infection of the sample by a canine circovirus.

23. An interfering RNA (iRNA) comprising at least 15 contiguous nucleotides complementary to the nucleic acid of claim 1 or 2.

24. A method for reducing the levels of a canine circovirus protein in an animal, viral mRNA in an animal or viral titer in a cell of an animal, the method comprising administering to the animal an iRNA of claim 23.

25. An isolated virus comprising at least 24 consecutive nucleotides from the nucleic acid of claim 1 or 2.

26. An isolated virus comprising at least 8 consecutive amino acids from the polypeptide of claim 5 or 6.
